(19) United States
(12) Patent Application Publication
(10) Pub. No.: US 2004/0268446 A1
(43) Pub. Date: Dec. 30, 2004

(54) Method for classifying plant embryos using Raman spectroscopy

(76) Inventors: Brian Penttila, Seattle, WA (US); Carolyn Carpenter, Seattle, WA (US)

Correspondence Address: WEYERHAEUSER COMPANY, INTELLECTUAL PROPERTY DEPT., CH 1J27, P.O. BOX 9777, FEDERAL WAY, WA, 98063, US

(21) Appl. No.: 10/853,483

(22) Filed: May 24, 2004

Related U.S. Application Data
(60) Provisional application No. 60/560,709, filed on Jun. 30, 2003.

(51) Int. Cl. A01H 1/00

(52) U.S. Cl. 800/295

(57) ABSTRACT

A three-step method for classifying plant embryo quality using Raman spectroscopy is provided. First, a classification model is developed based on Raman spectral data of reference samples of plant embryos or any portions of plant embryos of known embryo quality. The embryo quality may be known based on a comparison to a normal zygotic embryo or on actual planting of the embryo to observe its germination and subsequent growth. Then, a data analysis is carried out by applying one or more classification algorithms to the acquired Raman spectral data to develop a classification model. Second, Raman spectral data of a plant embryo or any portion of a plant embryo of unknown embryo quality are obtained. Third, the classification model developed in the first step is applied to the Raman spectral data obtained from the embryo (or any portions thereof) of unknown quality to classify the quality of this plant embryo.

### CROSS REFERENCE TO RELATED APPLICATION

The present application claims the benefit of U.S. Provisional Application No. 60/560,709, filed June 30, 2003.

### FIELD OF THE INVENTION

The invention is directed to classifying plant embryos to identify those embryos that are likely to successfully germinate and grow into normal plants, and more particularly, to a method for classifying plant embryos using Raman spectroscopy.

### BACKGROUND OF THE INVENTION

Reproduction of selected plant varieties by tissue culture has been a commercial success for many years. The technique has enabled mass production of genetically identical selected ornamental plants, agricultural plants and forest species. The woody plants in this last group have perhaps posed the greatest challenges. Some success with conifers was achieved in the 1970s using organogenesis techniques wherein a bud, or other organ, was placed on a culture medium where it was ultimately replicated many times. The newly generated buds were placed on a different medium that induced root development. From there, the buds having roots were planted in soil.

While conifer organogenesis was a breakthrough, costs were high due to the large amount of handling needed. There was also some concern about possible genetic modification. It was a decade later before somatic embryogenesis achieved a sufficient success rate so as to become the predominant approach to conifer tissue culture. With somatic embryogenesis, an explant, usually a seed or seed embryo, is placed on an initiation medium where it multiplies into a multitude of genetically identical immature embryos. These can be held in culture for long periods and multiplied to bulk up a particularly desirable clone. Ultimately, the immature embryos are placed on a development medium where they are intended to grow into somatic analogs of mature seed embryos. As used in the present description, a “somatic” embryo is a plant embryo developed by the laboratory culturing of totipotent plant cells or by induced cleavage polyembryogeny, as opposed to a zygotic embryo, which is a plant embryo removed from a seed of the corresponding plant. These embryos are then individually selected and placed on a germination medium for further development. Alternatively, the embryos may be used in artificial seeds, known as manufactured seeds.

There is now a large body of general technical literature and a growing body of patent literature on embryogenesis of plants. Examples of procedures for conifer tissue culture are found in U.S. Pat. Nos. 5,036,007 and 5,236,841 to Gupta et al.; U.S. Pat. No. 5,183,757 to Roberts; U.S. Pat. No. 5,464,769 to Attree et al.; and U.S. Pat. No. 5,563,061 to Gupta. Further, some examples of manufactured seeds can be found in U.S. Pat. No. 5,701,699 to Carlson et al., the disclosure of which is hereby expressly incorporated by reference. Briefly, a typical manufactured seed is formed of a seed coat (or a capsule) fabricated from a variety of materials such as cellulosic materials, filled with a synthetic gametophyte (a germination medium), in which an embryo surrounded by a tube-like restraint is received. After the manufactured seed is planted in the soil, the embryo inside the seed coat develops roots and eventually sheds the restraint along with the seed coat during germination. One of the more labor intensive and subjective steps in the embryogenesis procedure is the selective harvesting from the development medium of individual embryos suitable for germination (e.g., suitable for incorporation into manufactured seeds). The embryos may be present in a number of stages of maturity and development. Those that are most likely to successfully germinate into normal plants are preferentially selected using a number of visually evaluated screening criteria. A skilled technician evaluates the morphological features of each embryo embedded in the development medium, such as the embryo's size, shape (e.g., axial symmetry), cotyledon development, surface texture, color, and others, and selects those embryos that exhibit desirable morphological characteristics. This is a highly skilled yet tedious job that is time consuming and expensive. Further, it poses a major production bottleneck when the ultimate desired output will be in the millions of plants.

It has been proposed to use some form of instrumental image analysis for embryo selection to supplement or replace the visual evaluation described above. For example, PCT Application Serial No. PCT/US99/12128 (WO 99/63057), explicitly incorporated by reference herein, discloses a method for classifying somatic embryos based on images of embryos or spectral information obtained from embryos. Specifically, the method develops a classification model based on the digitized images or NIR (near infrared) spectral data of embryos of known embryo quality (e.g., potential to germinate and grow into normal plants, as validated by actual planting of the embryos and a follow-up study of the same or by the morphological comparison to normal zygotic embryos). The classification model is then applied to an image or spectral data of an embryo of unknown quality to classify the embryo according to its embryo quality.

While the use of NIR spectral data to assess the embryo quality has been successful in classifying embryos according to their quality, there is a continuing need to further refine the classification accuracy so as to identify only those embryos that are truly likely to germinate and grow into plants having various desirable characteristics. The present invention is directed to addressing this continuing need.

### SUMMARY OF THE INVENTION

The invention offers a method for classifying plant embryos according to their quality using Raman spectroscopy, including generally three steps. First, a classification model is developed. The classification model is developed first by acquiring Raman spectral data of reference samples of plant embryos of known embryo quality or any portions of such plant embryos. The embryo quality of these reference samples is known, for example, based on their comparison with normal zygotic embryos or based on actual planting of these embryos to observe their germination and subsequent growth into normal plants. Then, a data analysis is carried out by applying one or more classification algorithms to the acquired Raman spectral data to develop a classification model for classifying plant embryos by embryo quality. Second, Raman spectral data of a plant embryo of unknown embryo or any portions of such embryo are obtained. Third, the classification model developed in the first step is applied to the Raman spectral data obtained from the embryo of unknown quality (or any portions thereof) to classify the quality of the plant embryo.

According to one aspect of the present invention, Raman spectroscopy is used to identify the presence (and perhaps the quantity) of target analytes in an embryo that are indicative of the biochemical maturity of the embryo. For example, it has been determined that plant embryos that are biochemically matured so as to likely germinate and grow into normal plants include certain substances, such as sugar alcohols (e.g., pinitol, D-chiro-inositol, fagopyritol B1) and the raffinose series oligosaccharides (e.g., raffinose, stachyose). (See, U.S. Pat. Nos. 6,117,678 and 6,150,167 to Carpenter et al., which are explicitly incorporated herein by reference.) By identifying the presence of these target analytes, biochemically matured embryos suitable for incorporation into manufactured seeds can be identified.

The use of Raman spectroscopy to determine biochemical compositions of plant embryos permits further refinement of the classification of plant embryos according to their quality, so as to identify those embryos that are likely to germinate and grow into normal plants and hence are suitable for incorporation into manufactured seeds.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1 is a flowchart illustrating the steps of a method for classifying plant embryos using Raman spectroscopy, according to the present invention; and

FIG. 2 diagrammatically illustrates a tree embryo, wherein the circled areas indicate the embryo regions representative of the three embryo organs known as cotyledons, hypocotyl, and radicle.

### DETAILED DESCRIPTION OF THE PREFERRED EMBODIMENT

The present invention is directed to the use of Raman spectroscopy to assess biochemical maturity of plant embryos, such as conifer somatic embryos, to select those embryos suitable for further treatments such as incorporation into manufactured seeds.

Specifically, it has been determined that morphological features of an embryo alone, such as the embryo's size, shape (e.g., axial symmetry), cotyledon development, surface texture, color, and others, are not necessarily reliable predictors of the embryo's tendency to germinate. In other words, while certain morphological features of an embryo are necessary conditions for the embryo to successfully germinate, they are not sufficient conditions. The desirable embryo that is likely to germinate and grow into a normal plant must also be biochemically matured, which is difficult to assess based on the observation of the morphological features alone.

Raman spectroscopy, like NIR spectroscopy as employed in PCT Application Serial No. PCT/US99/12128 (WO 99/63057) discussed and incorporated by reference above, is a rapid non-invasive technique to identify and quantify analytes in complex samples. Briefly, a Raman spectrum is generated by illuminating a sample with a specific wavelength of light. The Raman spectrum, i.e., the scattered wavelengths and their relative intensities, are substance-specific to permit identification of a particular substance in the sample. Also, it is known that the intensity of Raman scattering is proportional to the number of molecules irradiated. Thus, Raman spectroscopy can be used to make both qualitative and quantitative measurements of analytes. Furthermore, Raman spectroscopy generally complements NIR spectroscopy, i.e., Raman spectroscopy can be used to identify analytes in an embryo that may not be identifiable with NIR spectroscopy. Therefore, the method of present invention provides reliable means to supplement NIR spectroscopy to further accurately assess embryos according to their quality. The theory and instrumentation of Raman spectroscopy are well known in the art, and therefore are not described in detail herein.

The present invention is directed to a method for classifying plant embryos according to their embryo quality using Raman spectroscopy. The embryo quality as used herein refers to one or more characteristics of an embryo that are susceptible to quantification to indicate whether the embryo is likely to successfully germinate and grow into a normal plant (and therefore, for example, be suited for incorporation into a manufactured seed). For example, the embryo quality includes the embryo's “conversion potential,” which means the capacity of a somatic embryo to germinate and grow in soil, preceded or not by desiccation or cold treatment of the embryo. The embryo quality may include further desirable characteristics, such as resistance to pathogens, drought resistance, heat and cold resistance, salt tolerance, resistance to lighting condition variation, etc. Embryos from all plant species can be evaluated according to the present inventive methods, while the methods have particular application to plant species where large numbers of somatic embryos are used to propagate desirable genotypes, such as forest tree species. In particular, the methods can be used to classify somatic embryos from conifer tree family Pinaceae, particularly from the genera: Pseudotsuga and Pinus.

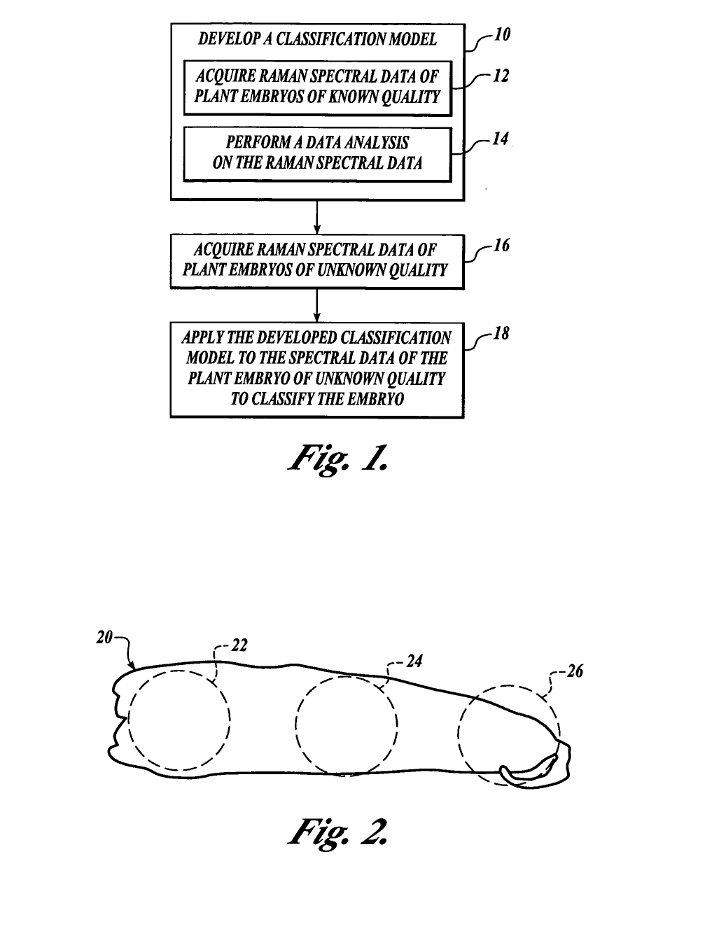

Referring to FIG. 1, a method of the present invention includes generally three steps. First, in step 10, a classification model is developed, as disclosure in PCT Application Serial No. PCT/US99/12128 (WO 99/63057) discussed and incorporated by reference above. Specifically, in sub-step 12, Raman spectral data are acquired from reference samples of plant embryos or any portions of plant embryos of known embryo quality. Referring additionally to FIG. 2, a plant embryo 20 has a well defined elongated bipolar structure including the three embryo organs known as cotyledons 22, hypocotyl 24, and radicle 26. Thus, Raman spectral data may be obtained from the embryo 20 as a whole, or from one or more of its portions 22, 24, 26, etc. The embryo quality of the reference embryos is known based on factual data, such as morphological or biochemical similarity to normal zygotic embryos or proven ability to germinate or convert to plants. In sub-step 14, the Raman spectral data acquired from the reference embryos or portions thereof are analyzed. Specifically, one or more classification algorithms are applied to the Raman spectral data. Essentially, the Raman spectral data from the reference embryos are used as the training set data to develop a classification model for classifying embryos by embryo quality. Second, in step 16, Raman spectral data of a plant embryo of unknown embryo quality or any portion of a plant embryo of unknown embryo quality are acquired. Third, in step 18, the classification model developed in the first step is applied to the Raman spectral data obtained in step 16, so as to classify the quality of the plant embryo. For example, embryos are classified based on how close their Raman spectral data fit to the classification model developed from the reference samples (the training set group).

Raman spectroscopy is highly suited for assessing the biochemical maturity of embryos. For example, biochemical maturity of an embryo can be determined based on the quantification of target analytes in an embryo, such as sugar alcohols (e.g., pinitol, D-chiro-inositol, fagopyritol B1) and the raffinose series oligosaccharides (e.g., raffinose, stachyose). (See, U.S. Pat. Nos. 6,117, 678 and 6,150,167 to Carpenter et al., which are explicitly incorporated herein by reference.) Further, biochemical maturity of an embryo can be assessed based on the quantification of various lipids such as triacylglycerides, and proteins such as dehydrins. Generally, dehydrins appear in an embryo for the first time during a later stage of embryo development, and therefore are good indicators of the embryo's biochemical maturity. Various known studies assert that embryo quality is related to gross chemical composition of the embryo or its parts, especially the amounts of water and storage compounds (proteins, lipids, and sugar alcohols and the raffinose series oligosaccharides as disclosed in the Carpenter et al. patents incorporated above). Raman spectroscopy provides a rapid, non-contact, and non-destructive method to quantify these and other target analytes in a plant embryo so as to classify embryos according to their biochemical maturity.

Further, Raman spectroscopy may be employed not to identify target analytes but to merely assess an embryo's general chemical composition. Specifically, because nearly all cell constituents of an embryo, including proteins, carbohydrates, lipids, nucleic acids, etc. produce Raman spectra, Raman spectroscopy can be used to acquire a “chemical image” of an embryo indicating the overall chemical composition of the embryo. Chemical images may be used, for example, to classify embryos as good (e.g., likely to germinate) or bad.

As well known in the art of spectroscopy, Raman spectra have rich information content. Oftentimes, Raman spectra have narrow sharp peaks that are relatively easy to isolate to identify any target analytes. Typically, acquired Raman spectra are used for chemical identification by matching the spectra with the spectra in pre-developed reference libraries. In this connection, it is noted that peaks for many analytes occur at identical locations, though of different signal intensities, in both Raman and mid-IR spectroscopic methodologies. Therefore, parallel analyses of Raman and mid-IR spectra may be helpful in associating certain spectral peaks with their corresponding analytes, and hence in developing the reference libraries.

Any suitable Raman spectroscopic instruments, including both dispersive instruments and FT (Fourier transform) based instruments, can be used. A suitable instrumentation includes an excitation light source (e.g., laser) to irradiate an embryo, a Raman sensor to collect a Raman scattering spectrum of the irradiated embryo, and a Raman data processor to process the collected Raman scattering spectrum. Generally, Raman spectroscopy instruments are available in the form of macro- or microscope based systems or fiber-optic probe based systems. For an in-process application, a fiber-optic probe based system may be more advantageous as it permits greater flexibility in interfacing the system with an embryo to be scanned. On the other hand, to address any low signal level or signal-to-noise ratio issues, directly coupled macro- and microscope based systems are more efficient in capturing the scattered photons. Microscope based systems may also be of value if the analytes of interest are non-uniformly distributed within an embryo. Specifically, if the analytes are more highly concentrated in localized regions of the embryo, they may be easier to detect at those regions. Depending on the size of these regions, microscope based systems may be more advantageous in scanning these regions of concentration because they typically have a finer spatial resolution than fiber-optic probe based systems. Measurement resolution is essentially dictated by the size of the exciting light (laser) spot. This is typically 50-100 micrometers in fiber-optic probes, and as small as 5-10 micrometers in the finest microscope systems.

When expected Raman signals are relatively weak, any suitable signal enhancement measures apparent to one skilled in the art may be used, such as RRS (Resonance Raman Spectroscopy) that generates an enhanced Raman signal when the analyte of interest has features which resonate with the irradiation (laser) wavelength. Also, if undesirable fluorescence from the sample (i.e., an embryo) is an issue, fluorescence can be minimized by moving the excitation laser wavelength into the red or infrared regions.

Preferably, each embryo or embryo region undergoes multiple light scans in order to obtain a representative average spectrum. In addition, multiple views of an embryo or embryo region, for example, the top view, the side view, and the end view of an embryo or embryo region, may be scanned to acquire further information on the embryo or embryo region. Also, for each embryo, multiple embryo regions (e.g., cotyledons, hypocotyl, and radicle) may be scanned in parallel or in sequence to refine and improve the classification accuracy.

The use of Raman spectroscopy to determine biochemical compositions of a plant embryo permits further refined classification of the embryo according to its quality, to identify those embryos that are likely to germinate and grow into normal plants and therefore are suitable for further treatments, such as incorporation into manufactured seeds.

While the preferred embodiments of the invention have been illustrated and described, it will be appreciated that various changes can be made therein without departing from the spirit and scope of the invention.

The embodiments of the invention in which an exclusive property or privilege is claimed are defined as follows:

1. A method for classifying plant embryo quality using Raman spectroscopy, comprising:
(a) developing a classification model by
(i) acquiring Raman spectral data of reference samples of plant embryos or any portions of plant embryos of known embryo quality;
(ii) performing a data analysis by applying one or more classification algorithms to the Raman spectral data, the data analysis resulting in development of a classification model for classifying plant embryos by embryo quality;
(b) acquiring Raman spectral data of a plant embryo or any portion of a plant embryo of unknown embryo quality; and
(c) applying the developed classification model to the Raman spectral data acquired in step (b) in order to classify the quality of the plant embryo of unknown embryo quality.

2. A method of claim 1, wherein the Raman spectral data acquired in step (a)(i) and (b) comprise data quantifying target analytes predetermined to indicate biochemical maturity of a plant embryo.

3. A method of claim 2, wherein the target analytes comprise sugar alcohols.

4. A method of claim 2, wherein the target analytes comprise lipids.

5. A method of claim 4, wherein the target analytes comprise triacylglycerides.

6. A method of claim 2, wherein the target analytes comprise proteins.

7. A method of claim 6, wherein the target analytes comprise dehydrins.

8. A method of claim 2, wherein the target analytes comprise the raffinose series oligosaccharides.

9. A method of claim 8, wherein the raffinose series oligosaccharides comprise a group consisting of raffinose and stachyose.

10. A method of claim 1, wherein the Raman spectral data are acquired in step (a)(i) and (b) from more than one view of the plant embryo or any portions thereof.

11. A method of claim 1, wherein the Raman spectral data are acquired in step (a)(i) and (b) from one or more embryo regions selected from the group consisting of cotyledons, hypocotyl, and radicle.

12. A method of claim 1, wherein the plant embryo quality is embryo conversion potential.

13. A method of claim 1, wherein the plant embryo is a plant somatic embryo.

14. A method of claim 1, wherein the plant is a tree.

15. A method of claim 14, wherein the tree is a member of the order Coniferales.

16. A method of claim 15, wherein the tree is a member of the family Pinaceae.

17. A method of claim 16, wherein the tree is selected from the group consisting of genera Pseudotsuga and Pinus.

18. A method of claim 17, wherein the tree is a loblolly pine.

19. A method of claim 18, wherein the Raman spectral data acquired in step (a)(i) and (b) comprise data quantifying target analytes predetermined to indicate biochemical maturity of a plant embryo, the target analytes comprising sugar alcohols consisting of pinitol, D-chiro-inositol, and fagopyritol B1.
